(19) United States
(12) Patent Application Publication
(10) Pub. No.: US 2011/0137388 A1
(43) Pub. Date: Jun. 9, 2011

(54) Heating support for patients

(76) Inventor: Axel Stihler, Stuttgart (DE)

(21) Appl. No.: 12/929,709

(22) Filed: Feb. 10, 2011

Related U.S. Application Data
(63) Continuation of application No. 12/000,796, filed on Dec. 18, 2007.

(30) Foreign Application Priority Data
Dec. 22, 2006 (DE) 10 2006 062 233.2

(51) Int. Cl. A61F 7/08

(52) U.S. Cl. 607/112

(57) ABSTRACT

A heating support (1) for a patient comprises a heating mat or a heating blanket (2) of a heat-insulating material, at least one heating element (3) that is integrated into the heating mat or heating blanket (2), and at least one temperature sensor (4). The heating support (1) is divided into a patient support area (7) suited for diagnostic imaging applications, and a temperature measurement area (9).

This application is a continuation of Ser. No. 12/000,796 filed Dec. 18, 2007 and also claims Paris Convention priority of DE 10 2006 062 233.2 filed Dec. 22, 2006 the complete disclosures of which are hereby incorporated by reference.

### BACKGROUND OF THE INVENTION

The present invention relates to an arrangement for controlling a heating support for a patient, which comprises a heating mat or heating blanket made of a heat-insulating material, at least one heating element that is integrated into the heating mat or heating blanket, and at least one temperature sensor, wherein the heating support is divided into a patient support area that is suitable for diagnostic imaging applications, and a temperature measurement area.

A heating support for patients of this type is disclosed e.g. in DE 10 2005 019 868 A1.

Heating supports for patients are used for medical examinations, operations and/or monitoring of patients who are in intensive care. Correct adjustment of the temperature of the heating support by the physician or the nursing staff is important. Towards this end, corresponding control means and temperature sensors that are connected to the control means are provided for controlling and adjusting the heating current that flows through the heating element.

It is thereby problematic that the temperature sensors and some heating elements that are used are not suited for diagnostic imaging applications, since they are not produced from a material that is transparent in accordance with these applications.

For this reason, the above-mentioned published document proposes not only to use a temperature sensor that is connected to the heating device but also to evaluate at least one further temperature signal related to a body feature of the patient for controlling the heating power. Measurement of the temperature on the skin surface of the patient is not very accurate and therefore rather unsuitable for controlling the heating device.

Temperature measurement on the patient is more demanding or interferes with the medical application.

It is therefore the underlying purpose of the invention to improve the conventional arrangement for controlling the heating support in such a fashion that handling is facilitated and the application of diagnostic imaging methods is not disturbed.

### SUMMARY OF THE INVENTION

This object is achieved by an arrangement in accordance with the independent claim. The temperature sensors in accordance with the invention are disposed in a measurement area which is displaced horizontally with respect to, outside of and not overlapping a patient support area. Accordingly, any radiation used for diagnostic imaging of the patient does not pass through the temperature sensors. This permits use of temperature sensors which are not transparent to the diagnostic imaging radiation used. Temperature measurement on the patient is thereby simulated in the temperature measurement area through the addition of insulating layers. For this reason, the remaining area of the heating support for a patient need not contain any temperature sensors.

By simulating the patient, the heating support for a patient can be adjusted to the patient without the patient being supported on the sensor field of the remaining support for a patient.

The temperature measurement area is advantageously completely integrated into the heating support for a patient.

For application of diagnostic medical imaging methods, the at least one heating element is formed by a carbon fiber.

Further advantageous further developments of the invention are described in the claims.

The drawing schematically shows one preferred embodiment of the invention, which is described below with reference to the figures of the drawing.

### BRIEF DESCRIPTION OF THE DRAWING

FIG. 1 shows a side view of a heating support;

FIG. 2 shows a top view of the heating support.

### DESCRIPTION OF THE PREFERRED EMBODIMENT

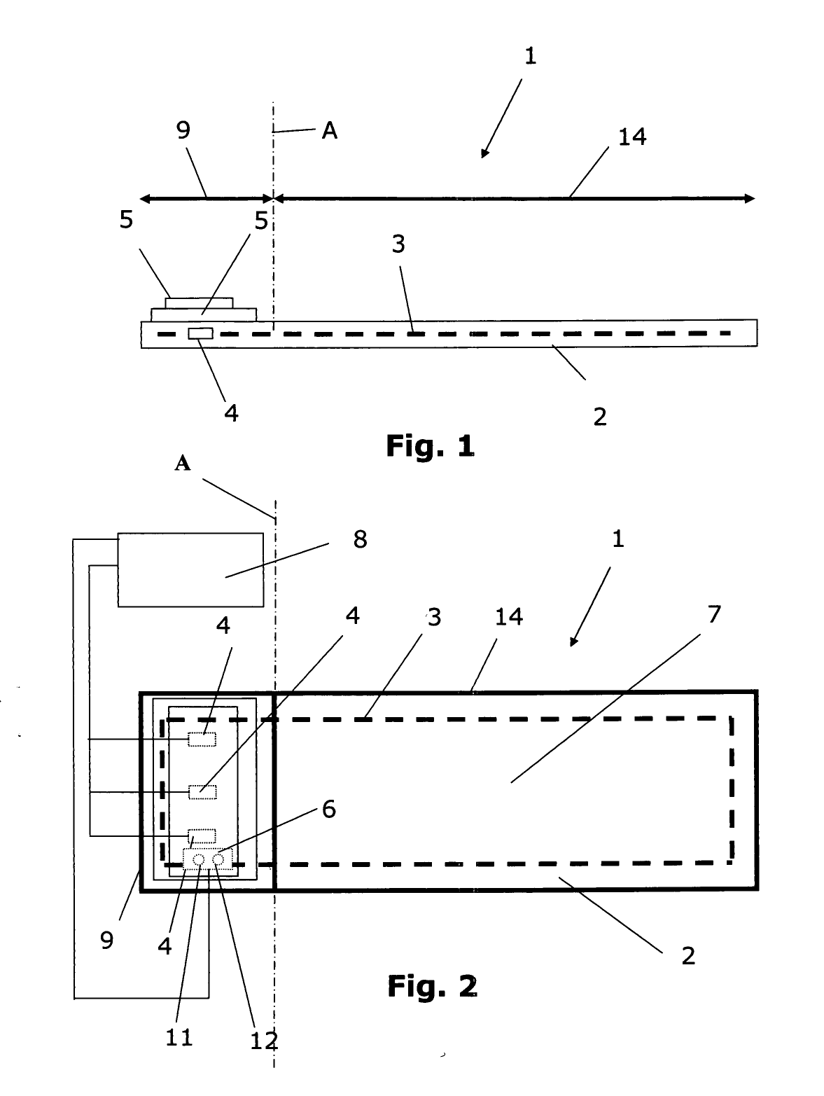

FIG. 1 shows that a heating support 1 comprises a heating blanket or a heating mat 2 into which a heating element 3 is integrated. The heating blanket or heating mat 2 is produced from a heat-insulating material. This may e.g. be a material that is distributed under the trademark thinsulate. Thinsulate™ insulation is a non-woven material that is used as heat insulation in textiles and bedding or also as acoustical insulation material in the automotive industry. The heating element 3 is formed by a conductor, such that the heating support 1 can be heated by a heating current. The heating element 3 may be designed as a carbon fiber which is transparent to X-rays and also has a low magnetic reduction, such that the heating element 3 can be used without any problem when the heating support 1 is used for diagnostic imaging applications. The heating support 1 is divided into an area 7 on which the patient to be examined is supported during the examination, and one area 9 that bears the electric connections 11, 12 as well as the temperature sensors 4 for controlling the heating support 1. NTC sensors are particularly suited as temperature sensors. NTC sensors are temperature-independent semi-conductor resistors whose conductivity is better at higher temperatures than at lower temperatures. For this reason, they are particularly suited for application in heating supports. One area 7, 14 is designed transparent for diagnostic imaging applications, while the other area 9 is not necessarily transparent due to the temperature sensors 4 and the electric connections 11, 12 for diagnostic imaging applications. Several layers of the heat-insulating material 5 are disposed above the temperature sensors 4. The number of layers of this material is adjusted in such a fashion that it is not necessary to measure the temperature in that area 7, 14 where the patient is supported. The several layers of the heat-insulating material 5 simulate a patient lying above the temperature sensors. Temperature measurement directly on the patient is therefore not necessary.

As is shown in FIG. 2, the heating element 3 is guided parallel to the edge of the heating support 1, wherein the electric connection 6, 11, 12 is formed on one side of the heating support 1. This produces a large transparent support area 7, 14 for diagnostic imaging applications. For defined control of the heating support 1, the temperature sensors 4 and the electric connection 6, 11, 12 are connected to a control unit 8 in order to control the heating current through the heating element 3 in such a fashion that a temperature of usually more than 30-39° C. is obtained for heating the patient. Measurement is thereby performed in that area 9 where the patient is not supported. The patient is simulated in this area by the layers of heat-insulating material 5. The control means 8 may comprise a data storage in which the characteristics for heating the patient are stored. The characteristics may be based on previous measurements, experience and a series of tests, which show which combination between number of layers of heat-insulating material 5 and heating current can be associated with a defined heating of the heating support 1. The several layers of heat-insulating material 5 thereby form a “standard patient”.

As clearly indicated in the figures, the heating and support device 1 has a patient support area 7, 14 in which the heating mat 2 is disposed. The heating mat 2 contains heat insulating material. The length and width of the patient support area 7 are suitable to warm and support a patient. For diagnostic imaging applications, the patient support area 7, 14 only comprises materials which are suitable for such applications. At least one heating element 3 is integrated into the heating mat 2 and extends from the patient support area 7, 14 into the temperature measurement area 9. The temperature measurement area 9 is displaced to the left in the figures and is separate from the patient support area 7, 14: there is no overlap between the temperature measurement area 9 and the patient support area 14. The temperature measurement area has at least one temperature sensor 4 as well as one layer or a plurality of layers of heat insulating material 5 is disposed in the temperature measurement area 9 above the temperature sensor 4. Through proper adjustment of the thickness and material used in the heat insulating layers 5, it is possible to calibrate the system in such a fashion that the temperature sensor 4 registers a temperature which corresponds to a temperature to which the patient is subjected in the patient support area 7, 14 when the patient lies on top of the heating mat 2. The displacement of the temperature sensors 4 into the temperature measure area 9 and away from the patient support area 7, 14 allows for diagnostic imaging applications on the patient without having any radiation used for such imaging passing through the temperature sensors 4. The temperature sensors 4 and the electrical connection 6, 11, 12 cannot interfere with the diagnostic images of the patient.

Accordingly, the heating support device 1 in accordance with the invention can be divided according to function into two areas, namely the patient support area 14 and the temperature measurement area 9. The vertical dashed line in FIGS. 1 and 2 labeled with reference symbol A symbolically indicates a separation demarcation between the temperature measurement area 9 and the patient support area 14. The heating mat 2 of the heating device 1 extends through both the patient support area 14 and the temperature measure area 9. The heating element 3 is disposed in the heating mat 2 and also extends within both the patient support area 14 as well as the temperature measurement area 9. The temperature sensors 4 located in the temperature measurement area 9 are disposed directly below the extra layers of insulation 5. Current is supplied to the heating element via first and second terminals 11 and 12, the first terminal 11 thereby supplying the current to the heating element 3 and the second terminal 12 removing the current from the heating element 3. Accordingly, the current supplied through the terminals 11 and 12 extends through both the temperature measurement area 9 as well as the patient support area 14.

As best seen in FIG. 1, the amount of insulation in heating mat 2 in the patient support area 7, 14 is substantially less than the overall amount of insulation in the combined heating mat 2 and additional heat insulating material layers 5 disposed directly above the temperature sensors 4 in the temperature measurement area 9. Since the heating element 3 extends through both the temperature support area 9 as well as the patient support area 7, 14, the presence of additional insulation 5 initially causes the temperature sensors 4 to measure a temperature which exceeds the temperature present on a surface of the heating mat 2 in the patient support area 7, 14, since heat loss through the reduced insulation present in the heating mat 2 in patient support area 7, 14 causes the upper surface of the mat to have a lower temperature in the patient support area 7, 14 than it does immediately below the extra insulating material layers 5 in the temperature measurement area 9. However, when a patient (not shown) is supported in the patient support area 7, 14 directly above the portions of the heating element 3 which extends through that patient support area 7, 14, the body warmth and insulating action of the patient result in an increase in the temperature of the heating mat 2 at the vertical surface interface between the heating mat 2 and the patient. Through proper selection of the material layers 5, the actual temperature felt by the patient in the patient support area 7, 14 can be precisely equal to the temperature measured by temperature sensor 4 in the temperature measurement area 9, although the temperature sensor 4 is not actually present in that patient support area 7, 14. The selection of the extra layers of heat insulating materials 5 can therefore be taken to effectively simulate the presence of the patient in the patient support area 7, 14. Accordingly, patient warming in diagnostic applications in which radiation passes through the patient support area 7, 14 can be effected using arbitrary sensors, since such sensors are not located in the imaging region and are consequently not part of the resulting diagnostic image. Nevertheless, the temperature measurement made by the temperature sensors 4 precisely tracks the actual temperature felt by the patient in the patient support area 7, 14, since the heat insulating material layers 5 have been calibrated to simulate the effect of the patient on the temperature at the upper surface of the heating mat 2 directly below the patient.

I claim:

1. A heating and support device for controlled warming of a patient, the device comprising:
a patient support area, said patient support area having a length and a width which is suitable to support and warm the patient;
a temperature measurement area, said temperature measurement area displaced horizontally with respect to, outside of and not overlapping said patient support area;
a heating mat having heat-insulating material, said heating mat extending throughout said patient support area and said temperature measurement area;
at least one heating element disposed at said heating mat, said heating element extending within said patient support area and said temperature measurement area;
at least one temperature sensor disposed in said temperature measurement area; and
several layers of heat-insulating material disposed in said temperature measurement area above said at least one temperature sensor, wherein said layers of heat-insulating material are disposed, structured and dimensioned to simulate the patient.

2. The device of claim 1, wherein three temperature sensors are provided in said temperature measurement area above which several layers of said heat-insulating material are disposed.

3. The device of claim 1, wherein said temperature sensor and said layers of heat-insulating material are integrated into said heating mat.

4. The device of claim 1 wherein said heating element is formed by a carbon fiber.

5. The device of claim 1, wherein said heating element heats both said temperature measurement area and said patient support area in a substantially uniform manner.

6. The device of claim 5, wherein a same heating current flows through both said temperature measurement area and said patient support area.

7. The device of claim 1, wherein said patient support area only comprises materials which are suited for diagnostic imaging applications.

8. The device of claim 1, wherein said temperature sensor comprises materials which are not transparent to diagnostic imaging radiation.

9. The device of claim 1, wherein said layers of heat insulating material are disposed directly above said temperature sensor.

10. The device of claim 1, wherein said patient support area contains no temperature sensor.
